(19) United States
(12) Patent Application Publication
(10) Pub. No.: US 2024/0277961 A1
(43) Pub. Date: Aug. 22, 2024

(54) CLOSED SUCTION AND OPEN SUCTION CATHETERS WITH MANUAL OPEN/CLOSE STOPCOCK TYPE VALVE

(71) Applicant: DRW Medical LLC, Aston, PA (US)

(72) Inventor: Robert Landis, Mountainside, NJ (US)

(21) Appl. No.: 18/170,833

(22) Filed: Feb. 17, 2023

(51) Int. Cl. A61M 16/04; A61M 1/00

(52) U.S. Cl. CPC A61M 16/0463; A61M 1/74

(57) ABSTRACT

A suction catheter apparatus for extracting secretions from a patient's airway including a suction collection tube, a catheter attached to the suction collection tube, and a control valve disposed between the catheter and the suction collection tube, the control valve set to an open position as the catheter is being inserted into the patient's airway while the suction is active so that the catheter extracts secretions as the catheter travels into the patient's airway to a predetermined depth. The suction catheter apparatus can be an open suction catheter apparatus or a closed suction catheter apparatus. This design feature provides a clear pathway for air flow from the atmosphere to reach a tip of the catheter instead of the air flow being extracted from the lungs of the patient.

### FIELD OF INVENTION

The present invention relates generally to medical devices, and more specifically, to a closed suction catheter and an open suction catheter with a manual open/close stopcock type valve.

### BACKGROUND

Patients in hospitals who are intubated and breathing through an endotracheal tube (ET) or tracheotomy tube (TT) need a clinician to maintain the ventilator and keep the ET tube or TT tube clear of secretions. A clinical standard for clearing secretions from patients ET or TT is the use of a Suction Catheter device.

One clinical standard is the use of a Closed Suction Catheter device which intersects between the patient's ET tube and the respiratory treatment device. The device has a plastic-bag-like sleeve around the catheter allowing for insertion into an ET tube without touching the catheter. The device uses a thumb-control valve that when pressed down activates the suction to the catheter. This system provides for the suction catheter to be repeatedly inserted and removed from the patient's airway without it being contaminated by outside sources. Another feature is that the suction catheter is readily available for insertion into the patient's airway without disconnecting the ventilator. It also provides for convenient repeated use of a disposable suction device without risk of cross contamination. Having the patient remain connected to the ventilator during suctioning maintains respiratory treatment with positive pressure and the needed respiratory flow rates.

Another clinical standard for clearing secretions from the patient's ET tube is the use of an Open Suction Catheter device. This catheter has a thumb-controlled chimney valve at the base of the catheter where room air is drawn in when the suction is connected and powered on. To activate the suction in the catheter, the user occludes the chimney valve with their thumb and the suction is diverted to the catheter drawing secretions and air from the patient's ET tube. The only time suction is applied to the catheter is when the chimney valve is occluded. When the chimney valve is open, suction in the catheter is off because the open chimney valve allows air into the suction tube.

Today's Suction Catheter devices are connected to a source of vacuum, such as hospital wall suction or a portable suction pump to extract secretions from a patient's ET tube. These types of vacuum sources lack flow rate control and have one common feature in that they all utilize a vacuum-filled container (the suction canister) which has a rigid wall that does not collapse under vacuum. The suction canister is connected to a vacuum pump or central vacuum source. During suctioning, secretions from the patient collect inside the canister, as the air flows out to the vacuum pump or suction wall outlet. During suctioning, there are two things that determine the amount of air flowing from the patient's airway, that is, the vacuum level inside the canister and the size and length of the suction tubing. Hospital wall suction systems and standard vacuum pumps cannot control suction flow. They have no method or feature to regulate the rate of air that flows from the patient's airway during suctioning.

Therefore, all present Suction Catheter devices are designed with the intention of being used with standard hospital wall suction or standard portable suction devices that cannot control flow rates. The control valves on current catheters are designed for use with uncontrolled suction flows. The suction collection tube connects to the vacuum filled canister on one end and to the suction catheter on the other end. When a Closed Suction Catheter is connected to the suction collection tube and the vacuum is turned on, the thumb control valve on the catheter is in a normally closed position. If it were not closed, there would be uncontrolled flow of air from the patient's breathing circuit into the suction canister. For example, the free flow of air through a 14 French catheter at 120 mmHg vacuum is ˜23 L/min. This rate of suction free flow would disrupt the breathing device treating the patient and may even cause collapse of the patient's lung. Therefore, all closed suction devices are designed with a normally closed thumb control valve where the suction collection tube is connected. This valve is spring loaded and opens to suction only when pushed down with a thumb and needs to be held down by the user during suctioning. When thumb pressure is released, the valve closes and suction stops.

### SUMMARY

In accordance with an embodiment, a closed suction catheter apparatus for extracting secretions from a patient's airway is provided. The closed suction catheter apparatus includes a suction collection tube, a catheter attached to the suction collection tube, and a control valve disposed between the catheter and the suction collection tube. The control valve is set to an open position as the catheter is being inserted into the patient's airway while the suction is active so that the catheter extracts the secretions as the catheter travels into the patient's airway to the predetermined depth.

In accordance with another embodiment, an open suction catheter apparatus for extracting secretions from a patient's airway is provided. The open suction catheter apparatus includes a suction collection tube, a catheter attached to the suction collection tube, and a control valve disposed between the catheter and the suction collection tube. The control valve is set to an open position as the catheter is being inserted into the patient's airway while the suction is active so that the catheter extracts the secretions as the catheter travels into the patient's airway to the predetermined depth.

In accordance with yet another embodiment, a method for extracting secretions from a patient's airway is provided. The method includes connecting a suction collection tube to a suction device at a catheter base, turning on suction and setting a control valve into an open position to make suction in the catheter active, inserting the active suction catheter into the patient's airway to clear secretions as the suction catheter advances to a predetermined depth, with the suction remaining active, withdrawing the catheter to clear remaining secretions from a full length of the patient's airway, for an open suction catheter, submerging a tip of the catheter into a flush solution to extract the flush solution through the catheter, and turning off a suction valve when the flush solution is extracted and the catheter is cleaned.

In accordance with yet another embodiment, a method for extracting secretions from a patient's airway is provided. The method includes connecting a suction collection tube to a suction device at a catheter base, turning on suction and setting a control valve into an open position to make suction in the catheter active, inserting the active suction catheter into the patient's airway to clear secretions as the suction catheter advances to a predetermined depth, with the suction remaining active, withdrawing the catheter to clear remaining secretions from a full length of the patient's airway, for a closed suction catheter, injecting a flush solution into a fitting at a tip of the catheter to extract the flush solution through the catheter, and turning off a suction valve when the flush solution is extracted and the catheter cleaned.

It should be noted that the exemplary embodiments are described with reference to different subject-matters. In particular, some embodiments are described with reference to method type claims whereas other embodiments have been described with reference to apparatus type claims. However, a person skilled in the art will gather from the above and the following description that, unless otherwise notified, in addition to any combination of features belonging to one type of subject-matter, also any combination between features relating to different subject-matters, in particular, between features of the method type claims, and features of the apparatus type claims, is considered as to be described within this document.

These and other features and advantages will become apparent from the following detailed description of illustrative embodiments thereof, which is to be read in connection with the accompanying drawings.

### BRIEF DESCRIPTION OF THE DRAWINGS

The invention provides details in the following description of preferred embodiments with reference to the following figures wherein:

FIG. 1 is an exemplary closed suction catheter with a manual open/close stop-cock type valve, in accordance with an embodiment of the present invention;

FIG. 2 is an exemplary open suction catheter with a manual open/close stop-cock type valve, in accordance with another embodiment of the present invention;

FIG. 3 is an exemplary method of employing Suction During Insertion (SDI) for an open suction catheter, in accordance with an embodiment of the present invention;

FIG. 4 is an exemplary method of employing SDI for a closed suction catheter, in accordance with an embodiment of the present invention; and

FIG. 5 is an exemplary block/flow diagram of the closed suction catheter with a manual open/close stopcock type valve, in accordance with an embodiment of the present invention.

Throughout the drawings, same or similar reference numerals represent the same or similar elements.

### DETAILED DESCRIPTION

Embodiments in accordance with the present invention provide for a closed suction catheter and an open suction catheter having a control valve, such as a stop-cock valve, that can be set and remain in an open position when suction is active. This enables Suction During Insertion (SDI) where the catheter extracts excretions as it is being inserted into a patient's airway. Thus, the user need not wait until a predetermined depth to commence extraction of excretions. Instead, the extraction of the secretions process can commence immediately upon initial insertion of the catheter (into the airway of a patient). The extraction of the secretions process thus takes place before reaching the predetermined depth, when the predetermined depth has been reached, and after reaching the predetermined depth, the catheter is slowly withdrawn clearing any remaining secretions. The advantage of immediately commencing the extraction of the secretions process during catheter insertion is that a clear pathway for the air flow from the atmosphere to reach the catheter tip is provided (instead of being extracted from the lungs). The supply of air from the atmosphere is sufficient to satisfy the controlled suction flow rate of a peristaltic pump, thereby minimizing the chance that air is being extracted from the lungs.

It is to be understood that the present invention will be described in terms of a given illustrative architecture; however, other architectures, structures, substrate materials and process features and steps/blocks can be varied within the scope of the present invention. It should be noted that certain features cannot be shown in all figures for the sake of clarity. This is not intended to be interpreted as a limitation of any particular embodiment, or illustration, or scope of the claims.

For some presently Closed Suction Catheter, the suction collection tube is connected to the closed suction device at the valve end. The suction is turned on and the thumb valve is normally closed. The catheter (without active suction) is inserted into the patient's airway to the prescribed or predetermined depth. Then the thumb valve is pressed to activate the suction and begin clearing secretions from the airways. As the thumb valve is pressed with suction active, the catheter is slowly withdrawn clearing secretions from the full length of the airway. When the catheter is out of the patient's airway the user releases the thumb valve and suction to the catheter is off. To clean the catheter a flush solution is injected into the fitting at the catheter tip as the thumb valve is activated to suck the flush solution through the catheter.

Those who are skilled with the use of closed catheter systems are aware that the only time suction is active in the catheter is when the thumb valve is actively pressed down into its open position.

For some presently Open Suction Catheter, the suction collection tube is connected to the Open Suction Catheter at the catheter base. The suction is turned on and the open chimney valve at the base of the catheter allows room air to be drawn in which keeps suction to the catheter off. The catheter (without suction) is inserted into the patient's airway to the prescribed or predetermined depth. Using their thumb, the chimney valve is occluded to activate suction in the catheter and begin clearing secretions from the airways. As the chimney valve is occluded with the suction active, the catheter is slowly withdrawn clearing secretions from the full length of the airway. When the catheter is out of the patient's airway the user opens the chimney valve and suction to the catheter is turned off. To clean the catheter, the tip of the catheter is submerged into a flush solution while the user occludes the chimney valve to suck the flush solution through the catheter.

Those skilled in the use of Open Suction Catheters are aware that the only time suction is active in the catheter is when the user occludes the open chimney valve.

Those skilled in the use of suction catheters and suction procedures recognize that presently the catheter is inserted into the patient without active suction. Then, when the catheter tip is inside the lung, suction is activated. Therefore, with the catheter tip deep into the patient's lungs when the suction starts, any air being extracted is likely to be from the lungs. This is because the distal end of the catheter is at the maximum distance from air supplied by the treatment device or the atmosphere. It is well known that air follows the path of least resistance, and, in this scenario, that path is likely air from the lungs. With today's standard catheter designs and procedures for suctioning, an excessive amount of air is being extracted from the lungs because the catheters and suction procedures are designed to be used with hospital wall suction and standard suction pumps which have no feature to regulate suction air flows. Extracting excessive amounts of air and oxygen from the patient's lungs during suctioning causes collapse of the alveoli leading to hypoxia and bradycardia, which can delay recovery and be an unpleasant experience for the patient.

The exemplary invention describes suction devices with a feature that supports a procedure for clearing airway secretions without extracting excessive air from the lungs. With the advent of suction pumps that work with flow control, there is a need for suction catheters with control features which are not presently available.

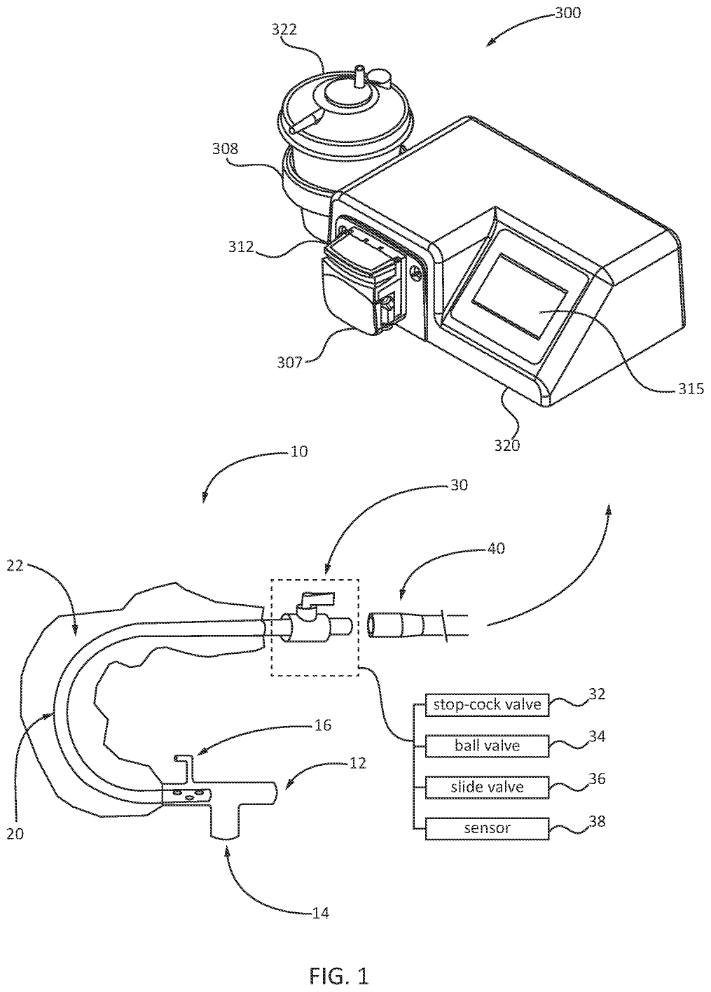

FIG. 1 is an exemplary closed suction catheter with a manual open/close stop-cock type valve, in accordance with an embodiment of the present invention.

The closed suction catheter 10 includes a catheter 20 covered by a protective sheath 22. At one end, the protective catheter sheath 22 has a fitting with three ports. One for an ET tube 12 to be connected thereto. The ET tube 12 connects to a patient (not shown). The protective catheter sheath fitting has a connector 14 and a flush port 16. The connector 14 connects to a ventilator (not shown). The other end of the catheter 20 is connected to a suction collection tube (or vacuum source) 40 via a manual open/close valve 30. The open/close valve 30 can be referred to as a control valve. The control valve 30 can be, e.g., a stop-cock valve 32, a ball valve 34 or a slide valve 36. The control valve 30 can further include one or more sensors 38.

The vacuum source 40 can be, e.g., a portable microprocessor-controlled peristaltic suction apparatus 300. The portable microprocessor-controlled peristaltic suction apparatus 300 includes a cover-display assembly 320. The cover-display assembly 320 has a display screen 315, as well as a pump head 307. The pump head 307 includes a flip top 312. The flip top 312 is shown in a closed configuration or closed state. Attached to the cover-display assembly 320 is the collection canister 308 and the bottom portion of the cover-display assembly 320. The control valve data can include SDI-related information, such as, e.g., suction variation as the catheter travels into the patient's airway, in a bidirectional manner.

Therefore, in accordance with FIG. 1, the closed suction catheter 10 allows an SDI procedure to be performed by one person (employing two hands). To perform an SDI procedure with conventional catheter systems employing a thumb activated valve, at least two people are involved (employing at least three hands). Regarding the closed suction catheter procedure, when a clinician inserts the catheter into the ET tube, it requires two hands. One hand to advance the catheter and the other hand to hold the ET Tube. The valve is closed. Once the catheter is at the desired depth, it takes two hands to withdraw it. One hand to hold the ET tube and the other hand to depress the valve while pulling the catheter back. To perform the SDI suction procedure with standard or conventional closed suction catheters requires three hands. A first hand to hold the valve down, a second hand to hold the ET tube, and a third hand to advance the catheter. In other words, two people are necessary to perform the SDI procedure. In contrast, the control valve 30 of the exemplary embodiments allows for one person to successfully perform the SDI procedure. Stated differently, the open/closed stopcock type valve 30 allows the SDI procedure with just two hands.

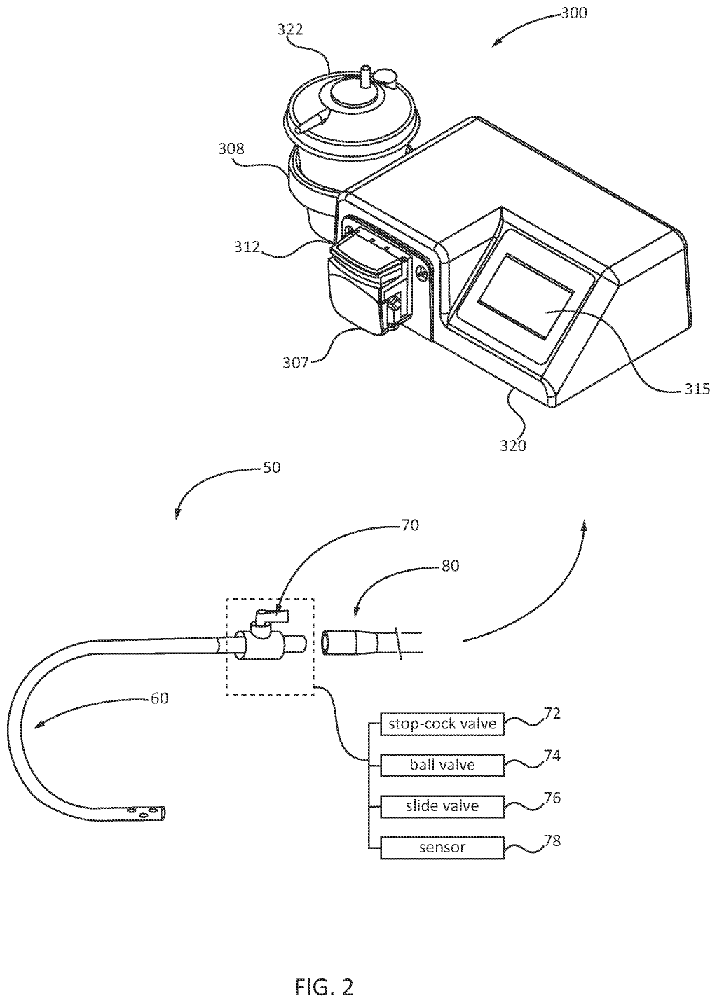

FIG. 2 is an exemplary open suction catheter with a manual open/close stop-cock type valve, in accordance with another embodiment of the present invention.

The open suction catheter 50 includes a catheter 60 connected to a suction collection tube (or vacuum source) 80 via a manual open/close valve 70. The open/close valve 70 can be referred to as a control valve. The control valve 70 can be, e.g., a stop-cock valve 72, a ball valve 74 or a slide valve 76. The control valve 70 can further include one or more sensors 78.

The vacuum source 80 can be, e.g., a portable microprocessor-controlled peristaltic suction apparatus 300. The portable microprocessor-controlled peristaltic suction apparatus 300 includes a cover-display assembly 320. The cover-display assembly 320 has a display screen 315, as well as a pump head 307. The pump head 307 includes a flip top 312. The flip top 312 is shown in a closed configuration or closed state. Attached to the cover-display assembly 320 is the collection canister 308 and the bottom portion of the cover-display assembly 320. The control valve data 70 can include SDI-related information, such as, e.g., suction variation as the catheter travels into the patient's airway, in a bidirectional manner.

The exemplary embodiments pertain to all patients in hospitals who are intubated, on ventilators breathing through an endotracheal tube (ET) or tracheotomy tube (TT) looking to recover and return to breathing through their normal airways. The exemplary invention aims to have the patient experience the suction procedure like someone just opened a window for a breath of fresh air.

The exemplary invention as described in FIGS. 1 and 2 presents a design feature for suction catheter devices making it more efficient at removing secretions with substantially less collateral air extraction from the lungs and minimizes the risk of lung collapse and hypoxia. It is noted that these catheter designs features were discovered when using the Microprocessor-Controlled Peristaltic Suction Apparatus 300 (U.S. application Ser. No. 17/826,610 filed May 27, 2022) incorporated herein by reference in its entirety. This type of suction pump is a positive displacement type pump which operates without a vacuum filled canister, eliminating the usual uncontrolled vacuum free flow. This type of suction pump controls suction flow and offers the opportunity to design suction catheters to compliment this advanced type of suction operation.

The exemplary invention as described in FIGS. 1 and 2 presents suction catheter devices with a control valve 30, 70 that can be open to suction without the user holding the valve open or occluding the open chimney valve. Instead of a spring-loaded thumb activated valve, this advanced style catheter has a stop-cock type valve, ball valve or slide valve that can be set and fixed into an open or closed position while suction is active. A person who is knowledgeable with manual open/closed valves for fluids recognizes the various design features for a valve with such function. The valve can be put into a fixed open position for suctioning, thus allowing the user to have it remain open. This design feature provides the user with a free hand for inserting the catheter into the patient's airway while suction is active. With this method of Suction During Insertion (SDI) the catheter is extracting secretions as it is being inserted into the patient's airway.

Extracting secretion as the closed catheter is being inserted into the ET tube has advantages. Secretions are cleared as the catheter is inserted into the ET tube thereby providing a clear pathway for the air flow from the treatment apparatus to reach the catheter tip. The supply of air from the treatment device is sufficient to satisfy the controlled suction flow rate of the peristaltic pump, thus minimizing the chance that air is being extracted from the lungs. The SDI procedure is an advancement for patient safety provided with this type of control valve on suction catheter devices. It is an advancement made safe because of the controlled suction flow feature inherent in Peristaltic Suction Devices. As described above, Closed Suction Catheters are connected in-line with a positive pressure treatment apparatus. The controlled suction flow does not disrupt the flow from such a treatment apparatus.

For the Open Suction Catheter with this design feature, the user has a free hand for inserting the catheter into the patient's airway while suction is active. With the SDI technique, the catheter is extracting secretions as it is being inserted into the patient's airway. Extracting secretions as the catheter is being inserted into the ET tube has advantages. Secretions are cleared as the catheter is inserted into the ET tube thereby providing a clear pathway for the air flow from the atmosphere to reach the catheter tip instead of being extracted from the lungs. The supply of air from the atmosphere is sufficient to satisfy the controlled suction flow rate of the peristaltic pump 300, thus minimizing the chance that air is being extracted from the lungs. The supply of air from the atmosphere to the catheter tip is unimpeded since the secretions have been cleared during insertion by using the SDI technique. Any air being extracted by the controlled suction flow rate of the peristaltic pump 300 is drawn unimpeded from the atmosphere leaving the air and oxygen in the patient's lungs.

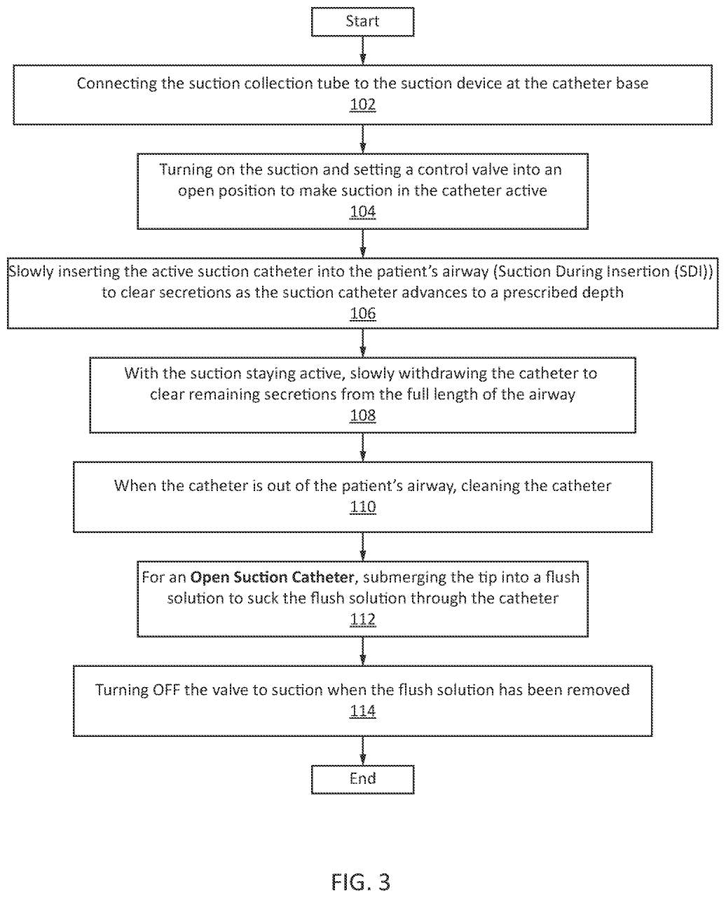

FIG. 3 is an exemplary method of employing Suction During Insertion (SDI) for an open suction catheter, in accordance with an embodiment of the present invention.

At block 102, connect the suction collection tube to the suction device at the catheter base.

At block 104, turn on the suction and set a control valve into an open position to make suction in the catheter active.

At block 106, slowly insert the active suction catheter into the patient's airway to clear secretions as the suction catheter advances to a prescribed or predetermined depth.

At block 108, with the suction staying active, slowly withdraw the catheter to clear remaining secretions from the full length of the airway.

At block 110, when the catheter is out of the patient's airway, clean the catheter.

At block 112, for an Open Suction Catheter, submerge the tip into a flush solution to suck the flush solution through the catheter.

At block 114, turn off the valve to suction when the flush solution has been removed.

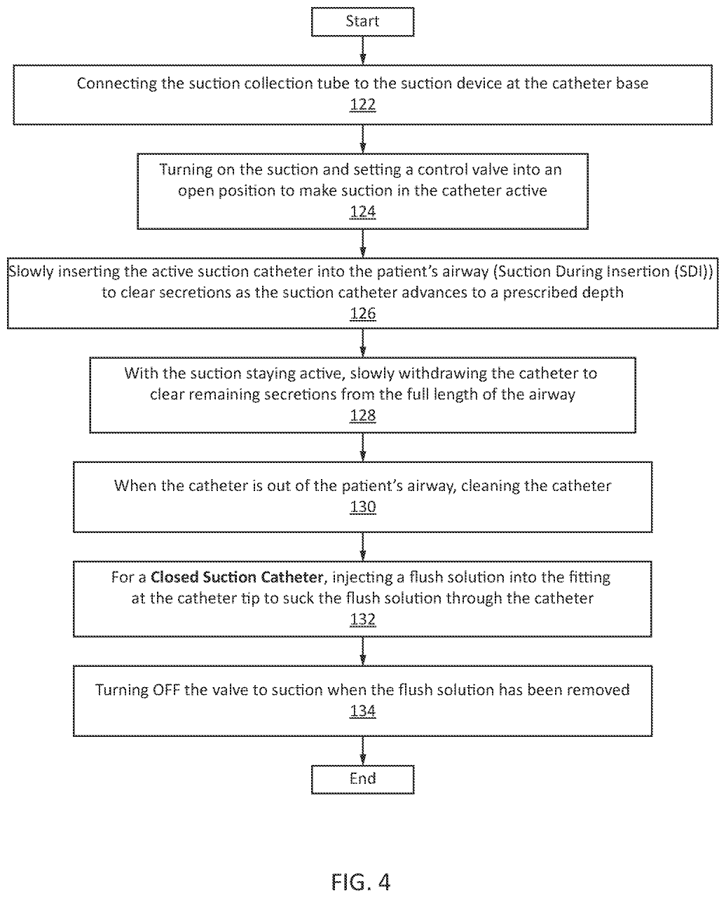

FIG. 4 is an exemplary method of employing SDI for a closed suction catheter, in accordance with an embodiment of the present invention.

At block 122, connect the suction collection tube to the suction device at the catheter base.

At block 124, turn on the suction and set a control valve into an open position to make suction in the catheter active.

At block 126, slowly insert the active suction catheter into the patient's airway to clear secretions as the suction catheter advances to a prescribed or predetermined depth.

At block 128, with the suction staying active, slowly withdraw the catheter to clear remaining secretions from the full length of the airway.

At block 130, when the catheter is out of the patient's airway, clean the catheter.

At block 132, for a Closed Suction Catheter, inject a flush solution into the fitting at the catheter tip to suck the flush solution through the catheter.

At block 134, turn off the valve to suction when the flush solution has been removed.

Therefore, for the SDI procedure, the suction collection tube is connected to the suction device at the catheter base. The suction is turned on and the valve is set into an open position making suction in the catheter active. The active suction catheter is slowly inserted into the patient's airway (SDI) clearing secretions as it advances to the prescribed or predetermined depth. Because of the controlled suction flow rate of the peristaltic pump, any air the catheter is extracting can easily be drawn through the cleared airway from the treatment device or the atmosphere, rather than the lungs (path of least resistance). With the suction staying active, the catheter is slowly withdrawn clearing remaining secretions from the full length of the airway. When the catheter is out of the patient's airway, the user goes to clean the catheter.

To clean the Open Suction Catheter, the tip of the catheter is submerged into a flush solution to suck the flush solution through the catheter. When completed, the valve to suction is turned off.

To clean the Closed Suction Catheter, a flush solution is injected into the fitting at the catheter tip to suck the flush fluid through the catheter. When completed, the valve to suction is turned off.

With reference to FIGS. 1-4, the exemplary suction catheter devices are configured for use with positive displacement suction pumps that have control over suction flow rates when extracting secretions. The exemplary invention discloses advanced suction catheters with design features to accommodate SDI procedures, thereby reducing the amount of air and oxygen being sucked out of the patient's lungs during suction procedures.

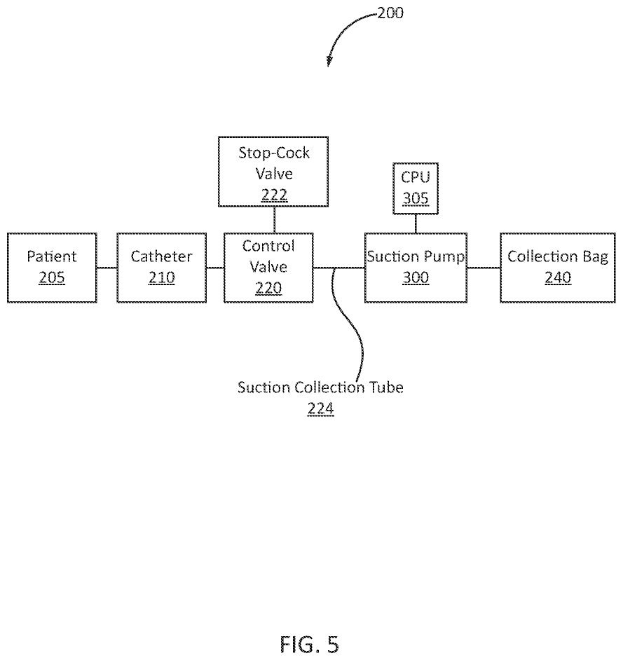

FIG. 5 is an exemplary block/flow diagram of the closed suction catheter with a manual open/close stopcock type valve, in accordance with an embodiment of the present invention.

The system 200 includes a catheter 210 inserted into a patient 205, the catheter having a control valve 220, such as a stop-cock valve 222. The control valve 220 is connected to, e.g., a suction pump 300 via a suction collection tube 224. The suction pump 300 includes one or more CPUs 305. The suction pump 300 mechanically cooperates with a collection bag 240 to receive secretions extracted from the patient 205 via the catheter 210.

In conclusion, in conventional systems, there is an unnecessary amount of air that is evacuated when the thumb control valve of the catheter is depressed, (even before secretion clearance begins). When the thumb control valve is initially pressed, there is no substantial extraction of secretions. However, the suction is on at the max limit, which causes high vacuum at the tip of the catheter. When the thumb control valve is depressed to commence extraction of secretions, large amounts of air are sucked out and there is a very high flow at that instant (since the suction was on). As a result, air is sucked out too fast that may cause negative issues in the patient's lungs. The exemplary embodiments alleviate such issues by providing for a closed suction catheter and an open suction catheter having a control valve, such as a stop-cock valve, that can remain in an open position when suction is active. This enables SDI where the catheter extracts excretions as it is being inserted into a patient's airway. Thus, the user need not wait until a predetermined depth to commence extraction of excretions. Instead, the extraction of excretions process can commence immediately upon initial insertion of the catheter (at the airway of the patient). The extraction of excretions process thus takes place before reaching the predetermined depth, when the predetermined depth has been reached, and after reaching the predetermined depth, the catheter is slowly withdrawn clearing any remaining secretions.

The advantage of using the control valves described herein is that the closed suction catheter allows an SDI procedure to be performed by one person (employing two hands). To perform an SDI procedure with conventional catheter systems employing a thumb activated valve, at least two people are involved (employing at least three hands). Regarding the closed suction catheter procedure, when a clinician inserts the catheter into the ET tube, it requires two hands. One hand to advance the catheter and the other hand to hold the ET Tube. The valve is closed. Once the catheter is at the desired depth, it takes two hands to withdraw it. One hand to hold the ET tube and the other hand to depress the valve while pulling the catheter back. To perform the SDI suction procedure with standard or conventional closed suction catheters requires three hands. A first hand to hold the valve down, a second hand to hold the ET tube, and a third hand to advance the catheter. In other words, two people are necessary to perform the SDI procedure. In contrast, the control valves of the exemplary embodiments allow for one person to successfully perform the SDI procedure.

Moreover, the advantage of immediately commencing the extraction of excretions process is that a clear pathway for the air flow from the atmosphere to reach the catheter tip is provided (instead of being extracted from the lungs). The supply of air from the atmosphere is sufficient to satisfy the controlled suction flow rate of the peristaltic pump, thus minimizing the chance that air is being extracted from the lungs. Moreover, there is no sudden high air flow once the catheter tip reaches the predetermined depth because suctioning of secretions has already commenced as the catheter has travelled to the predetermined depth. Thus, air is not suctioned out too fast at any specific point in the process, which could have a negative impact on a patient's lungs (as when a thumb control valve is employed).

While there have been shown, described and pointed out fundamental novel features of the present principles, it will be understood that various omissions, substitutions and changes in the form and details of the methods described and devices illustrated, and in their operation, may be made by those skilled in the art without departing from the spirit of the same. For example, it is expressly intended that all combinations of those elements and/or method steps which perform substantially the same function in substantially the same way to achieve the same results are within the scope of the present principles. Moreover, it should be recognized that structures and/or elements and/or method steps shown and/or described in connection with any disclosed form or implementation of the present principles may be incorporated in any other disclosed, described or suggested form or implementation as a general matter of design choice. It is the intention, therefore, to be limited only as indicated by the scope of the claims appended hereto.

It should also be understood that the example embodiments disclosed and taught herein are susceptible to numerous and various modifications and alternative forms. Thus, the use of a singular term, such as, but not limited to, “a” and the like, is not intended as limiting of the number of items. Furthermore, the naming conventions for the various components, functions, parameters, thresholds, and other elements used herein are provided as examples, and can be given a different name or label. The use of the term “or” is not limited to exclusive “or” but can also mean “and/or”.

Having described preferred embodiments, which serve to illustrate various concepts, structures and techniques that are the subject of this patent, it will now become apparent to those of ordinary skill in the art that other embodiments incorporating these concepts, structures and techniques may be used. Additionally, elements of different embodiments described herein may be combined to form other embodiments not specifically set forth above.

Accordingly, it is submitted that that scope of the patent should not be limited to the described embodiments but rather should be limited only by the spirit and scope of the following claims.

1. A closed suction catheter apparatus for extracting secretions from a patient's airway, the closed suction catheter apparatus comprising:
a suction collection tube;
a catheter attached to the suction collection tube; and
a control valve disposed between the catheter and the suction collection tube, the control valve set to an open position as the catheter is being inserted into the patient's airway while suction is active so that the catheter extracts the secretions as the catheter travels into the patient's airway to the predetermined depth.

2. The closed suction catheter apparatus of claim 1, wherein the control valve is a stop-cock type valve.

3. The closed suction catheter apparatus of claim 1, wherein the control valve is a ball valve.

4. The closed suction catheter apparatus of claim 1, wherein the control valve is a slide valve.

5. The closed suction catheter apparatus of claim 1, wherein the suction collection tube is connected to a peristaltic suction pump.

6. The closed suction catheter apparatus of claim 1, wherein a clear pathway for air flow is provided from the atmosphere to reach a tip of the catheter instead of the air flow being extracted from lungs of the patient.

7. An open suction catheter apparatus for extracting secretions from a patient's airway, the open suction catheter apparatus comprising:
a suction collection tube;
a catheter attached to the suction collection tube; and
a control valve disposed between the catheter and the suction collection tube, the control valve set to an open position as the catheter is being inserted into the patient's airway while suction is active so that the catheter extracts the secretions as the catheter travels into the patient's airway to the predetermined depth.

8. The open suction catheter apparatus of claim 7, wherein the control valve is a stop-cock type valve.

9. The open suction catheter apparatus of claim 7, wherein the control valve is a ball valve.

10. The open suction catheter apparatus of claim 7, wherein the control valve is a slide valve.

11. The open suction catheter apparatus of claim 7, wherein the suction collection tube is connected to a peristaltic suction pump.

12. The open suction catheter apparatus of claim 7, wherein a clear pathway for air flow is provided from the atmosphere to reach a tip of the catheter instead of the air flow being extracted from lungs of the patient.

13. A method for extracting secretions from a patient's airway, the method comprising:
connecting a suction collection tube to a suction device at a catheter base;
turning on suction and setting a control valve into an open position to make suction in the catheter active;
inserting the active suction catheter into the patient's airway to clear secretions as the suction catheter advances to a predetermined depth;
with the suction remaining active, withdrawing the catheter to clear remaining secretions from a full length of the patient's airway;
for an open suction catheter, submerging a tip of the catheter into a flush solution to extract the flush solution through the catheter; and
turning off a suction valve when the flush solution is completely extracted.

14. The method of claim 13, wherein the control valve is a stop-cock type valve.

15. The open suction catheter apparatus of claim 13, wherein the control valve is a ball valve.

16. The open suction catheter apparatus of claim 13, wherein the control valve is a slide valve.

17. The open suction catheter apparatus of claim 13, wherein the suction collection tube is connected to a peristaltic suction pump.

18. A method for extracting secretions from a patient's airway, the method comprising:
connecting a suction collection tube to a suction device at a catheter base;
turning on suction and setting a control valve into an open position to make suction in the catheter active;
inserting the active suction catheter into the patient's airway to clear secretions as the suction catheter advances to a predetermined depth;
with the suction remaining active, withdrawing the catheter to clear remaining secretions from a full length of the patient's airway;
for a closed suction catheter, injecting a flush solution into a fitting at a tip of the catheter to extract the flush solution through the catheter; and
turning off a suction valve when the flush solution is completely extracted.

19. The method of claim 18, wherein the control valve is a stop-cock type valve.

20. The method of claim 18, wherein the suction collection tube is connected to a peristaltic suction pump.
